(19) United States
(12) Patent Application Publication
(10) Pub. No.: US 2022/0174887 A1
(43) Pub. Date: Jun. 9, 2022

(54) POLLINATOR ATTRACTION POT

(71) Applicant: Daniel Ager, Nicasio, CA (US)

(72) Inventor: Daniel Ager, Nicasio, CA (US)

(21) Appl. No.: 17/545,750

(22) Filed: Dec. 8, 2021

Related U.S. Application Data
(60) Provisional application No. 63/122,887, filed on Dec. 8, 2020.

(51) Int. Cl. A01G 9/02; A01H 1/02

(52) U.S. Cl. CPC A01G 9/026; A01H 1/027

(57) ABSTRACT

A pollinator attraction pot includes a pot body made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot. A pollinator attractor is positionable on or within the pot body. The pollinator attractor includes a material or substance that serves to attract a pollinator to the pollinator attraction pot.

### PRIORITY

The present application claims the benefit of domestic priority based on U.S. Provisional Patent Application 63/122,887 filed on Dec. 8, 2020, the entirety of which is incorporated herein by reference.

### BACKGROUND

Pots have been used for centuries for growing plants. Such pots have been made out of a variety of materials, including plastic, clay, terracotta, stone, cast iron, and metal. Each material has different water holding characteristics.

Recently, fabric pots have been becoming more and more ubiquitous. Fabric pots can be designed to allow a predetermined amount of permeability to both air and water. One particular fabric gaining prominence is a non-woven geotextile fabric. The non-woven geotextile fabric reduces root circling and promotes air pruning. Air pruning allows for faster plant growth and higher harvest yields.

Many plants require pollination for reproduction and the production of seeds or fruit. Pollination is the act of transferring pollen grains from the male anther of a flower to the female stigma. Seeds can only be produced when pollen is transferred between flowers of the same species. Flowers must rely on vectors to move pollen. Vectors can include wind, water, birds, insects, and other animals. Animals and insects are often referred to as pollinators. Pollination usually occurs as a result of a pollinator's unintended interaction with or proximity to a flower. Thus, it is in a flower's interest to attract pollinators. Flowers can encourage the attraction of pollinators by their color, scent, or the like. However, heretofore there have not been pots that assist in the attraction of pollinators.

There is therefore a need for a pot that assists in the attraction of pollinators to a plant contained within the pot. There is further a need for a pot with a pollinator attractor that is an integrated part of the pot. There is further a need for a pot with a pollinator attractor that is a structural component of the pot.

### SUMMARY

The present invention satisfies these needs. In one aspect of the invention, an improved pot is provided.

In another aspect of the invention, a pollinator attraction pot is provided.

In another aspect of the invention, a pollinator attraction pot includes an integrated pollinator attractor.

In another aspect of the invention, a pollinator attraction pot includes a pollinator attractor that serves as a structural component of the pot.

In another aspect of the invention, a kit is provided that allows for the attraction of selected pollinators to a pot.

In another aspect of the invention, a method of attracting a pollinator comprises growing a plant in a pot and providing on the pot a pollinator attractor.

In another aspect of the invention, a method of attracting a pollinator comprises growing a plant in a pot and selecting a pollinator attractor from a group of pollinator attractors. The present invention satisfies these needs.

In another aspect of the invention, a pollinator attraction pot comprises a pot body made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot; and a pollinator attractor positionable on or within the pot body, wherein the pollinator attractor includes a material or substance that serves to attract a pollinator to the pollinator attraction pot.

In another aspect of the invention, a pollinator attraction pot comprises a pot body made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot; and a pollinator attractor positionable on or within the pot body, wherein the pollinator attractor includes a material or substance that serves to attract a pollinator to the pollinator attraction pot, and wherein the pollinator attractor is an integral part of the pot body.

In another aspect of the invention, a pollinator attraction pot comprises a pot body made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot; and a pollinator attractor positionable on or within the pot body, wherein the pollinator attractor includes a material or substance that serves to attract a pollinator to the pollinator attraction pot, and wherein the pollinator attractor is a visual stimulator that is selected to attract a pollinator, wherein the visual stimulator is a color that reflects light at a predetermined wavelength.

In another aspect of the invention, a pollinator attraction pot comprises a pot body made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot; and a pollinator attractor positionable on or within the pot body, wherein the pollinator attractor includes a material or substance that serves to attract a pollinator to the pollinator attraction pot, and wherein the pollinator attractor comprises a thread woven into the pot body.

In another aspect of the invention, a method of attracting a pollinator to a plant comprises planting a plant in a pot body, the pot body being made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot; and positioning a pollinator attractor on or within the pot body, wherein the pollinator attractor includes a material or substance that serves to attract a pollinator to the pollinator attraction pot.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1A is a pollinator attraction pot according to one version of the invention;

FIG. 1B is a pollinator attraction pot according to another version of the invention;

FIG. 1C is a pollinator attraction pot according to another version of the invention;

FIG. 2A is a pollinator attraction pot having a spring mechanism according to a version of the invention;

FIG. 2B is a pollinator attraction pot having a spring mechanism according to another version of the invention;

FIG. 2C is a pollinator attraction pot having a spring mechanism according to another version of the invention;

FIG. 3A is a pollinator attraction pot according to another version of the invention; and

FIG. 3B is a pair of different sized pollinator attraction pot according to another version of the invention.

### DESCRIPTION

The present invention relates to a pot for growing a plant. In particular, the invention relates to a pot for growing a plant wherein the pot includes a pollinator attractor. Although the invention is illustrated and described in the context of being useful for fabric pots, and the like, the present invention can be useful in other instances. Accordingly, the present invention is not intended to be limited to the examples and embodiments described herein.

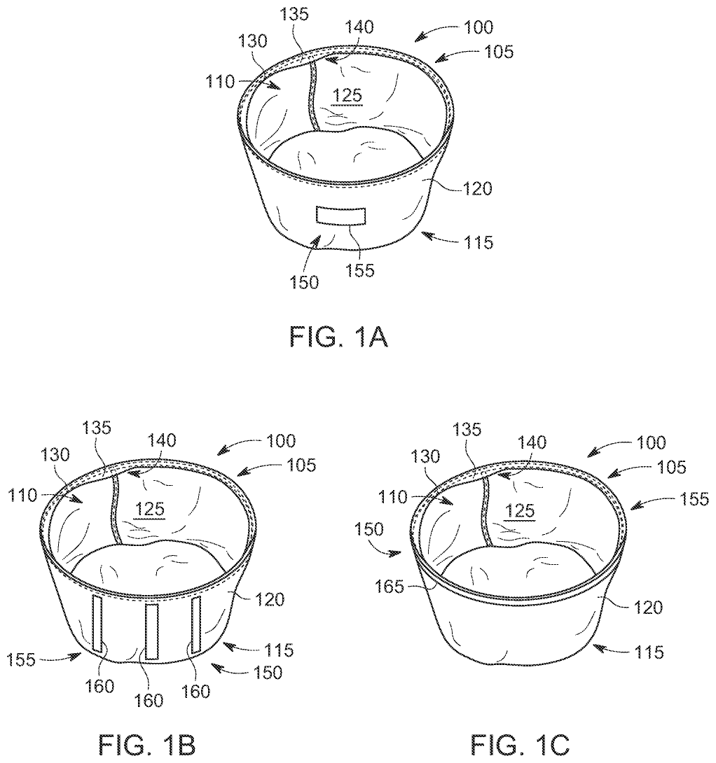

FIG. 1A shows a pollinator attraction pot 100 according to a version of the invention. The pollinator attraction pot 100 has a pot body made up of a top portion 105 having an opening 110, a bottom portion 115, and a side wall 120 connecting the top portion 105 and the bottom portion 110. In the version shown in FIG. 1A, the top portion 105 and opening 110 are circular, the bottom portion 115 is circular, and the side wall is correspondingly cylindrical with a circular cross-section. However, in other versions, the top portion 105, opening 110, bottom portion 115, and side wall 120 can take on any other geometrical shape. The bottom portion 115 and sidewalls 120 define an interior 125, and the opening 110 provides access to the interior 125.

The side wall 120 can be made of a material that is suitable to serve as a plant pot. In one version, the side wall 120 can be made of a flexible material. By flexible material, it is meant that the material can be bent, folded, or otherwise similarly manipulated by a user's hands without the need for excessing force being applied. In one version, the flexible material is a flexible fabric material that can be sewn or otherwise pieced together in a fabric-like manner. In one version, the side wall 120 is sufficiently rigid to not collapse on its own yet sufficiently bendable to able to fold over. In another version, the side wall 120 is made to collapse under its own weight. The bottom portion 115 can be made of the same or a different material. The side wall 120 material is capable of holding soil so that a plant can be planted in the soil. The side wall 120 material may also be able to hold various other materials.

In one version, the side wall 120 and/or the bottom portion 115 are water permeable. By water permeable it is meant that water can pass through the material within a reasonable amount of time under normal planting conditions. Examples of water permeable, flexible fabric materials include one or more of a woven or non-woven geotextile fabric, a polymer, a polymer-based fabric, either biodegradable or non-biodegradable, a plant-based fabric material, such as cotton, hemp, burlap, jute, bamboo, paper/cardboard composite, PLA, biodegradable composite material, and the like. In one particular version, the side wall 120 comprises a woven or non-woven geotextile fabric.

In one version, the side wall 120 and/or the bottom portion 115 are water impermeable. By water impermeable it is meant that the material is able to retain a significant portion of water within the pot body for a period of time, such as a day. For the water-impermeable version, examples of materials for the side wall 120 and/or bottom portion include one or more of polymer, plastic such as polypropylene, polyester, polyethylene, PVC, and/or ABS, leather or other animal based biproduct, cellulose based material, and the like.

Additional features can be provided for the pot body. For example, the side wall 120 can have handles or can have no handles. Also, the side wall 120 can be made of a rigid material, such as metal or clay.

In one particular version, the pollinator attraction pot 100 can be in the form of a collapsible container. The top portion 105 can be held open by a rim member 130 that is sized and shaped to extend around the perimeter or circumference of the opening 110. The rim member 130 can be a wire, tube, rod, or the like, and is sufficiently rigid to maintain the opening 110 in an open configuration so that the interior 125 can be accessed. The rim member 130 can be a wire, tube, rod, or the like made of one or more of metal, fiberglass, biodegradable stick, wicker, paper, cardboard, bamboo, plant based material, polymer, plastic, such as PVC and/or polyethylene, composites and the like and may be a single piece of material or multiple pieces, may be may be braided, stranded, flat, round or other shape, and may be coated or uncoated. The top portion 105 includes a flap 135 of material that creates a pocket 140 around the top of the interior of the collapsible container 100. The rim member 130 is containable within the pocket 140. The rim member 130 when inside the pocket 140 maintains a desired shape and thus maintains the opening 105 in the desired shape, such as the open position or configuration. In the version shown in FIG. 1A, the rim member 130 is a circular wire, and it maintains a circular opening as shown. Alternatively, the rim member may have a different shape, such as oval, ovoid, ovate, egg-shaped, square, rectangular, other polygon, star, heart, other decorative shape, or any other shape. The side wall 120 can have a shape that corresponds shape to the opening 110 and rim member 130 or can have a different shape. One or more fasteners, such as a snap, button, Velcro, zipper, zip-lock, or the like, can be provided to fasten the flap 135 to the side wall 120 to retain the rim member 130 in the pocket 140. Specific examples of suitable collapsible containers are described in U.S. patent application Ser. No. 16/676,120 filed on Nov. 6, 2019, the entirety of which is incorporated herein by reference.

The pollinator attraction pot 100 includes a pollinator attractor 150. The pollinator attractor 150 is a component positionable on, near, or within the pollinator attraction pot 100, and most preferably on or within. The pollinator attractor includes any material or substance that serves to attract a pollinator to the pollinator attraction pot 100. By pollinator attractor it is meant any material or substance that is designed so that when positioned on or near the pot makes the pot more attractive to a pollinator than the pot would be in the absence of the pollinator attractor. Pollinators include animals, such as insects, that serve as vectors for the transfer of pollen from one flower of a species to another flower of the same species. For example, insects, such as bees, are very efficient pollinators for many types of plants. Pollination usually occurs as a result of a pollinator's unintended interaction with or proximity to a flower. Thus, it is in a plant's interest to attract pollinators. By providing a pollinator attractor 150 as part of the pollinator attraction pot 100, a plant growing in the pot has a greater chance of being pollinated than one in a pot lacking a pollination attractor 150. The pollinator attractor 150 can be an integral part of the pollinator attraction pot 100, such as by being permanently or securely attached thereto, or can be a separable element selectively attachable the pot or positionable near the pot. For example, the pollinator attractor pot 100 can include a pocket or other similar part that can receive the pollinator attractor 150.

The material or substance of the pollinator attractor 150 can vary depending on the specific pollinator that is desired to be attracted. Examples of pollinator attractors 150 include aromatic stimulators, such as scents, and visual stimulators, such as certain colors or wavelengths of reflected light.

In one version, the pollinator attractor 150 can comprise a material that includes and/or emits a chemical scent that attracts a pollinator. Specific universal examples include peppermint, lavender, basil, and oregano. Bees are also particularly attracted to rosemary, sage, thyme, chamomile, marjoram, and bee balm. It is also believed that caffeine can be an effective bee attractant. Commercially available bee attractants are also available. In another version, the pollinator attractor 150 can comprise a material that detracts a pollinator rather than attracts the pollinator. For some plants, pollination is not desired. For example, cannabis has female plants with flowers that lose value when pollinated and produce seeds.

In one version, the pollinator attractor 150 is a visual stimulator that is selected to attract a pollinator. Visual stimulation can occur by the reflection of certain wavelengths of light that are reflected from the pollinator attractor 150 to an observing pollinator. One manner of providing a visual stimulation to a pollinator is by providing the pollinator attractor 150 with a color that reflects a predetermined wavelength at a predetermined reflectivity. For example, the pollinator attractor 150 can reflect light at wavelength of from about 635 nm to about 700 nm (red), from about 590 nm to about 635 nm (orange), from about 560 nm to about 590 nm (yellow), and/or from about 400 nm to about 450 nm (violet). Each wavelength or combination of wavelengths can be selected to be associated with a particular pollinator that is suitable for a particular plant and/or suitable for a particular climate or geography.

In one version, a pollinator attraction pot 100 can be provided with the ability to attract multiple pollinators and/or can have the ability to select the type of pollinator from a plurality of options. For example, a plurality of pollinator attraction pots 100 can be provided with each of the pollinator attraction pots 100 having a different pollinator attractor 150. In this way, a user can select the pollinator attraction pot 100 to use depending on the plant to be grown in the pot and/or the pollinator that is desired to be attracted. For example, a first pollinator attraction pot 100 can be equipped with a first colored pollinator attractor 150 associated with a first pollinator and a second pollinator attraction pot 100 can be equipped with a second colored pollinator attractor 150 that is associated with a second pollinator. For example, bees are attracted to bright blue and violet colors. Hummingbirds prefer red, pink, fuchsia, or purple flowers. Butterflies are attracted to bright colors such as yellow, orange, pink, and red. Birds often tend to be attracted to reds. In another version, a pollinator attraction pot 100 can be equipped with a plurality of different pollinator attractors 150.

As shown in FIG. 1A, the pollinator attractor 150 can be in the form of a strip 155 of material that is or that contains a material or substance that is attractive to a pollinator. For example, the strip 155 of material can be entirely a material that is attractive to a pollinator. Alternatively, the strip 155 can be a piece of material, such as a fabric, that has a material or substance that a pollinator is attracted to attached to or incorporated within the pot body material, such as by adhesive, sewing, hook and loop fastener, or by otherwise being integrally incorporated. The strip 155 may be positioned anywhere on the pollinator attraction pot 100, such as on the outside of the side wall 120 as shown in FIG. 1A. In FIG. 1B, the strip 155 is takes the form of one or more vertical strips 160 on the outside of the side wall 120. When multiple vertical strips 160 are provided, each vertical strip 160 can contain the same pollinator attractor substance or a different pollinator attractor substance. In FIG. 1C, the strip 155 is shown as a band of top strip 165 of material positioned at the top portion 105 near the opening 110 of the pollinator attraction pot 100. In any of these version, the strip 155 can be a separable strip of material or can merely be a portion of the pot. For example, for a fabric pot, the strip 155 can be any portion of the fabric pot to which a pollinator attractor is attached or integrated into.

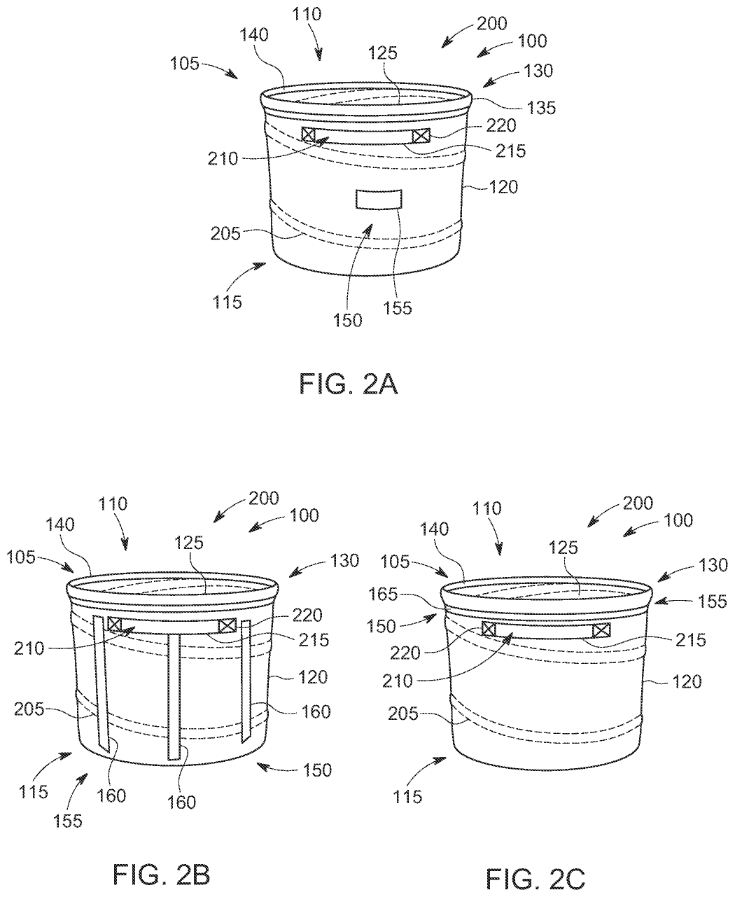

Another version of pollinator attraction pot 100 is shown in FIG. 2A. In this version, the pollinator attraction pot 100 takes the form of a spring-loaded collapsible pot 200. The spring-loaded collapsible pot 200 has a skeletal spring 205 contained within or on the side wall 120. The skeletal spring 205 is biased upwardly and serves to maintain the pot in an open configuration. The skeletal spring 205 can also be collapsed and secured in a collapsed configuration by a Velcro strap or the like to maintain the pot in a collapsed configuration. One or more handles 210 can also be provided to help carry the pot, especially when loaded with a plant and/or dirt. In the version shown, the handle 210 can include a strap 215 of material attached to the side wall 120 at a connection 220. Versions of collapsible fabric plant pots are disclosed in U.S. patent application Ser. No. 13/457,921 filed on May 9, 2012 and in U.S. patent application Ser. No. 15/330,075 filed on Aug. 3, 2016, both of which are incorporated herein by reference in their entireties.

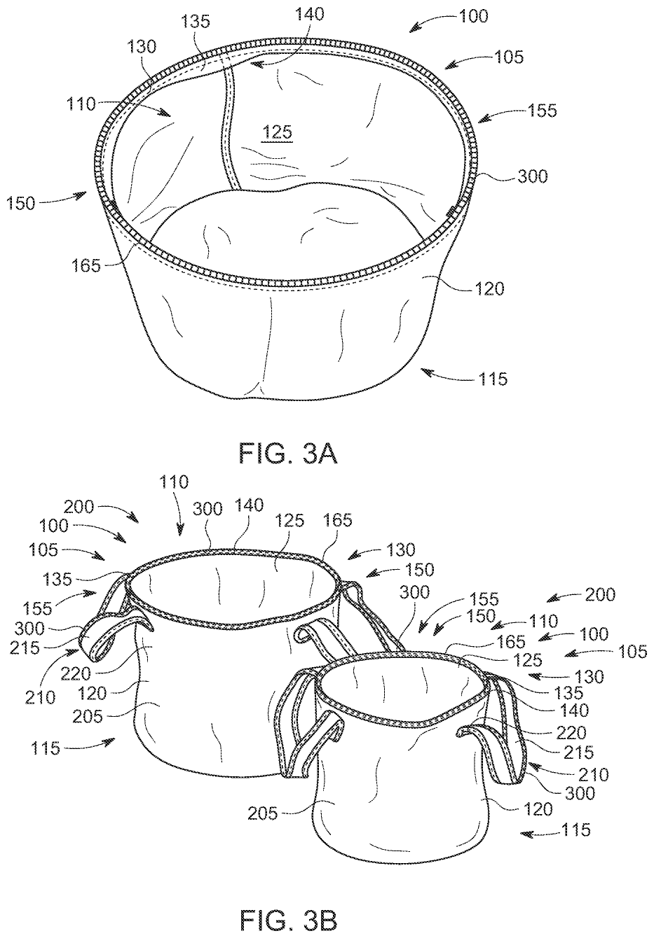

FIG. 3A shows a particular version of a pollinator attraction pot 100 of the invention. In this version, the pollinator attractor 150 is in the form of a colored thread 300. The colored thread 300 can be woven into a fabric side wall 120 of the pot merely for the purpose of serving as a pollinator attractor 150 or can be used as the thread of the pot or a portion of the pot that serves to also structurally hold the pot together. For example, as shown in FIG. 3A, the colored thread 300 can be used to sew the top of the pollinator attraction pot 100 together at or near the top opening 110. FIG. 3B shows the colored thread 300 being used with a spring-loaded pot 200. In the version of FIG. 3B, the colored thread 300 is also shown as being on or near the top of the pot near the opening 110. In addition or alternatively, the colored thread 300 can be provided on the handle 210 along the strap 215 and/or at the connection 220. FIG. 3B also shows different sized pollinator attraction pots 100, each provided with colored thread 300.

The colored thread 300 can be selected to advantageously both attract a pollinator and secure the pot together. The colored thread 300 can comprise any suitable material, such as cotton, nylon, and/or the like. The colored thread 300 can be UV treated to maintain the color after exposure to sunlight.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number, and all directional limitations, such as up and down and the like, can be switched, reversed, or changed as long as doing so is not prohibited by the language herein with regard to a particular version of the invention. Also, the various features of the versions herein can be combined in various ways to provide additional versions of the present invention. Furthermore, certain terminology has been used for the purposes of descriptive clarity, and not to limit the present invention. Throughout this specification and any claims appended hereto, unless the context makes it clear otherwise, the term “comprise” and its variations such as “comprises” and “comprising” should be understood to imply the inclusion of a stated element, limitation, or step but not the exclusion of any other elements, limitations, or steps. Throughout this specification and any claims appended hereto, unless the context makes it clear otherwise, the term “consisting of” and “consisting essentially of” and their variations such as “consists” should be understood to imply the inclusion of a stated element, limitation, or step and not the exclusion of any other elements, limitations, or steps or any other non-essential elements, limitations, or steps, respectively. Throughout the specification, any discussed on a combination of elements, limitations, or steps should be understood to include a disclosure of additional elements, limitations, or steps and the disclosure of the exclusion of additional elements, limitations, or steps. All numerical values, unless otherwise made clear in the disclosure or prosecution, include either the exact value or approximations in the vicinity of the stated numerical values, such as for example about +/− ten percent or as would be recognized by a person or ordinary skill in the art in the disclosed context. The same is true for the use of the terms such as about, substantially, and the like. Also, for any numerical ranges given, unless otherwise made clear in the disclosure, during prosecution, or by being explicitly set forth in a claim, the ranges include either the exact range or approximations in the vicinity of the values at one or both of the ends of the range. When multiple ranges are provided, the disclosed ranges are intended to include any combinations of ends of the ranges with one another and including zero and infinity as possible ends of the ranges. Therefore, any appended or later filed claims should not be limited to the description of the preferred versions contained herein and should include all such alterations, permutations, and equivalents as fall within the true spirit and scope of the present invention.

What is claimed is:

1. A pollinator attraction pot comprising:
A pot body made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot; and
a pollinator attractor positionable on or within the pot body,
wherein the pollinator attractor includes a material or substance that serves to attract a pollinator to the pollinator attraction pot.

2. A pollinator attraction pot according to claim 1, wherein the pollinator attractor is an integral part of the pot body.

3. A pollinator attraction pot according to claim 1, wherein the pollinator attractor is permanently attached to the pot body.

4. A pollinator attraction pot according to claim 1, wherein the pollinator attractor emits a chemical scent.

5. A pollinator attraction pot according to claim 1, wherein the pollinator attractor is a visual stimulator that is selected to attract a pollinator.

6. A pollinator attraction pot according to claim 1, wherein the pollinator attractor is a visual stimulator that is selected to attract a pollinator, wherein the visual stimulator is a color that reflects light at a predetermined wavelength.

7. A pollinator attraction pot according to claim 6, wherein the predetermined wavelength is from 635 nm to 700 nm.

8. A pollinator attraction pot according to claim 6, wherein the predetermined wavelength is from 590 nm to 635 nm.

9. A pollinator attraction pot according to claim 6, wherein the predetermined wavelength is from 560 nm to 590 nm.

10. A pollinator attraction pot according to claim 6, wherein the predetermined wavelength is from 400 nm to 450 nm.

11. A pollinator attraction pot according to claim 1, wherein the pollinator attractor comprises multiple, different pollinator attractors.

12. A pollinator attraction pot according to claim 1, wherein the pollinator attractor comprises a strip of material affixed to the side wall.

13. A pollinator attraction pot according to claim 1, wherein the pollinator attractor comprises a band of material on the top portion.

14. A pollinator attraction pot according to claim 1, wherein the pollinator attractor comprises a thread woven into the pot body.

15. A pollinator attraction pot according to claim 14, wherein the thread is part of the construction of the flexible fabric material.

16. A pollinator attraction pot according to claim 1, wherein the top portion and the bottom portion are circular and wherein the sidewall is at least partially cylindrical.

17. A pollinator attraction pot according to claim 1, wherein the flexible fabric is water permeable.

18. A pollinator attraction pot according to claim 1, wherein the flexible fabric is water impermeable.

19. A pollinator attraction pot according to claim 1, wherein the pot body is collapsible.

20. A method of attracting a pollinator to a plant, the method comprising:
planting a plant in a pot body, the pot body being made up of a top portion having an opening, a bottom portion, and a side wall connecting the top portion and the bottom portion, the bottom portion and side wall defining an interior which can be used to contain a growing plant, wherein the sidewall is made of a flexible fabric material suitable to serve as a plant pot; and
positioning a pollinator attractor on or within the pot body,
wherein the pollinator attractor includes a material or substance that serves to attract a pollinator to the pollinator attraction pot.
